(19) United States
(12) Patent Application Publication
(10) Pub. No.: US 2026/0086089 A1
(43) Pub. Date: Mar. 26, 2026

(54) DEVICE, METHOD AND SYSTEM FOR DIAGNOSING, MONITORING AND PREDICTING CHRONIC HEALTH CONDITIONS THROUGH URINE BIOMARKERS

(71) Applicant: Herstasis Health Foundation, New Westminster (CA)

(72) Inventors: Kathryn FINK, New Westminster (CA); Jennifer THOMPSON, New Westminster (CA); Jennifer VANDER ZALM, New Westminster (CA)

(21) Appl. No.: 19/112,944

(22) PCT Filed: Sep. 15, 2023
(86) PCT No.: PCT/CA2023/051227

§ 371 Date: Mar. 18, 2025

Related U.S. Application Data
(60) Provisional application No. 63/408,995, filed on Sep. 22, 2022.

(51) Int. Cl. G01N 33/543; A61B 10/00

(52) U.S. Cl. CPC G01N 33/54388; A61B 10/007; G01N 2333/575; G01N 2333/59

(57) ABSTRACT

A device, system and method for measuring and monitoring analyte concentrations in urine. The device is a cassette comprising two or more hormone or metabolite fluorometric or colorimetric assays, one of which can quantify urine dilution or concentration relative to population standards. The cassette can accommodate multiple lateral flow and/or dry chemistry assays, wherein the fluorometric or colorimetric results are readable quantitatively by a countertop or handheld reader and the results are normalized by the urine concentration measurement. In preferred embodiments, the analytes relate to symptoms of menopausal transition and have dynamic ranges sufficient to handle the known analyte fluctuations during such transition.

### FIELD OF THE INVENTION

The present invention relates to measurement of multiple analyte concentrations in urine. More specifically, the present invention relates to a device, method and system for measuring hormone and/or metabolite concentrations related to a chronic condition in a user's urine, and for providing results thereof to said user.

### BACKGROUND

At-home measurement of hormones and metabolites in bodily fluids (urine, saliva, etc.) is readily available in the form of qualitative and semi-qualitative colorimetric lateral flow assays (over-the-counter pregnancy tests, FSH test strips, COVID-19 rapid antigen tests, etc.), quantitative reader-assisted lateral flow assay (Mira™ Fertility, Inne™, ClearBlue™ Advanced Fertility monitor) and colorimetric urinalysis strips for basic urine properties (pH, specific gravity, glucose, protein, bilirubin, etc.). Urine testing allows assessment of both free hormones and hormone metabolites, which can provide a fuller picture of physiological processes. However, concentration measurements are significantly affected by the water content of urine. Therefore, this type of testing focuses on identification of significant changes, such as the order-of-magnitude increases in analytes such as human chorionic gonadotropin (hCG) during pregnancy or luteinizing hormone (LH) just prior to ovulation. Determining analyte concentration without normalizing for urine concentration may be sufficient for identification of major trends-such as spikes an order of magnitude or larger related to ovulation. However, variability in urine concentration can mask or falsely indicate in hormone & metabolite secretion. Studies that assess day-to-day intra- and inter-individual patterns require testing the urine at a central laboratory in order to quantify and correct for water content via normalization with creatinine or specific gravity. Expanding the capacity and capability of urine testing to monitor chronic conditions changing on a daily or weekly basis therefore requires the ability to correct for water composition. It is thus contemplated that a device, method and system that provides for hormone or metabolite assays (e.g. fluorometric or colorimetric), in combination with functionality to normalise the analyte concentrations to account for fluctuations in urine concentration would be desirable.

### BRIEF SUMMARY OF THE INVENTION

It is envisioned that the present invention may address or help to address a number of problems. Frequent, normalized at-home hormone measurement paired with daily symptom logging and pattern tracking algorithms may provide benefits related to symptom prediction, trigger identification and evaluating the success of medical and lifestyle interventions.

It is contemplated that the present invention will find particular application as a device, method and system for measurement and monitoring of menopause-related symptoms, and the present invention is primarily discussed and illustrated herein in the context of a device for measurement of analytes (hormones and/or metabolites) relating to menopause transition. However, it is contemplated that the device may also be applied/adapted for measurement of other analytes and/or relating to other conditions. For example, prostate cancer screening via urine quantification of two or more urinary biomarkers, e.g., pigment epithelium-derived factor, hemopexin, calnexin precursor, hornerin, keratin 13, etc., has a demonstrably higher performance than traditional measurement of prostate-specific antigen in serum. It is contemplated that multivariate assessment of urinary hippurate, formate and 4-cresol sulfate can be used to distinguish between inflammatory bowel disease, Crohn's disease and ulcerative colitis, with implications for diagnosis and disease management. Repeat measurements of urinary N-telopeptide in the early menopause transition allows for prediction of osteopenia prior to significant lumbar spine bone loss, enabling early intervention. Other examples of conditions and their corresponding suitable analytes are provided in Table 1 below.

The present invention provides a means to manage and predict/measure menopause symptoms through personal pattern finding and analysis with accurate at-home hormone and metabolite testing, with analytes relevant to the menopausal transition, normalized for fluctuations in urine concentration. It is contemplated that longitudinal hormone and metabolite data, combined with qualitative symptom data, individual anthropometrics and socioeconomics would provide a means to reduce the cost and burden for diverse population women's health research studies and permit more efficient use of health care resources through education and personalized data.

50% of the human population experiences the menopausal transition as they reach mid-life. Despite the negative impacts that menopausal transition can have on quality of life, work life, and healthcare costs, there are pervasive issues with the standard of care for menopausal symptoms. These issues stem from several different sources, including lack of physician training, widespread confusion about the risks and benefits of hormone therapy and the relative dearth of alternative proven therapies. The current clinical practice for diagnosing menopause itself—defined as 12 months since the last menstrual period—is by definition retrospective.

Symptom Prediction. There are known correlations between at least 10 hormones and disruptive perimenopausal symptoms, including hot flushes, sleep and mood disruption, brain fog, body pain, headaches, incontinence and low libido. Identification of hormonal changes as they happen informs the user of possible impending symptom changes and enables them to prepare for such events.

Trigger Identification. In addition to hormone changes, perimenopause symptoms can be worsened or triggered on an individual basis due to other events such as high stress events, consumption of certain food, beverages, or intoxicants, a change in physical activity level, etc. Daily symptom logging and longitudinal data visualization allows retrospective analysis of potential triggering events, providing future actionable data.

Intervention Efficacy. Clinical guidelines for hormone therapy suggest the minimum effective dose and duration of use. A majority of North American people who experience menopause consider alternative treatments such as natural health products (“NHPs”) to manage their symptoms. There is little evidence of the widespread efficacy of NHPs, with some indications that individual variables including biometrics, diet, and genetics may impact NHP success. Hormone tracking with daily symptom logging provides improved cost/benefit clarity for success of both hormone therapy and alternative treatments including NHP, yoga, meditation etc.

Menopause Prediction. Multiple studies suggest that predicting the likely duration of an individual's menopausal transition confers several benefits, including a “light at the end of the tunnel” for when undesirable symptoms might cease, better information for contraceptive practices, and an estimation of the length of use of hormone therapy. A prospective diagnosis requires longitudinal analysis of age, bleeding regularity, alongside frequent (>monthly) measurement of hormone (e.g., follicle-stimulating hormone (FSH) and estrogen (E2)) and hormone metabolites (e.g., pregnanediol, a progesterone metabolite) levels, all of which are captured and analyzed as a part of this method.

Women's Health Research. Best practices for clinical investigations of therapeutic products—including medical devices, pharmaceuticals, and natural health products—advise that such studies be designed to identify whether there are differences by sex that affect the safety and efficacy of the products. Hormones related to female fertility cyclically fluctuate over time-scales ranging from days to weeks, from puberty to a decade or more after the final menstrual period, and are one known causes of differences in reaction by sex. This highly quantitative system detects and tracks changes in relevant hormones in a highly accessible manner, supporting greater inclusivity in clinical trials while providing a means to assess how hormone changes impact safety and efficacy.

Improved Efficiency of Healthcare Resources. Mid-life women experiencing menopause symptoms have higher medical, pharmacy, and sick leave costs relative to controls, imparting a burden both at the individual and societal level. Menopause symptoms are typically chronic-persisting over months or years-rather than acute, often requiring long-term management or treatment adjustments. The hormones underlying many of these symptoms vary by day and week, so routine monitoring of treatment and intervention efficacy by traditional laboratory testing is costly, invasive, and with a multi-day or multi-week delay to results, is retrospective. This invention, especially when paired with symptom and biometric tracking, provides a quantitative method to easily and inexpensively monitor symptom and hormone trends outside of the clinical laboratory. The significant increase in physician and patient adoption of home health monitoring systems over the past years further supports the adoption of managing chronic symptoms outside of the clinic settings

Necessity of Normalization of Urine Concentration Measurements. Excretion of biomarkers such as hormones and metabolites by the kidneys into urine is physiologically separate from the amount of water in the urine. The latter is driven largely by hydration status and extrarenal water losses, which, as further discussed below, can vary inter- and intra-individually on an hourly and daily basis. However, in the absence of a correcting or normalizing factor, the amount of water in the urine significantly dictates the measured concentration of any urine analyte; as an example, doubling the urine volume by drinking water will halve the measured concentration of a urinary biomarker. Many at-home urine tests, such as those used for testing fertility, make the assumption that for a specific individual, collection at a consistent time of day will produce fairly consistent urine compositions.

Some aspects/features of the present invention may include:
Adaptable for use with various types of analytes (e.g. hormones and hormone analytes with known correlations to perimenopausal symptoms).
Analyte detection ranges (dynamic ranges to cover known correlations with perimenopausal symptoms).
Normalization method: Integrated quantitative method to normalize analyte concentrations relative to changes in urine concentration. The normalization step captures actual production & excretion trends by the endocrine system in order to accurately describe changes related to perimenopausal symptoms and the menstrual cycle.
Fluorescent multiplex lateral flow assays for simultaneous detection of multiple analytes.
Single-use cassette to house 2 or more colorimetric and fluorometric assays for simultaneous detection of analytes from urine collected on an exposed sample pad, and an identifying mark or feature for assay lot and calibration curve information.
Countertop reader unit capable of detection and concentration conversion of, at minimum, visual and fluorescent labels, having internal memory for short-term data storage. The countertop reader unit is provided with Wi-Fi or other wireless communication capability for sharing, communicating or receiving data.
Mobile device application with data acquisition and processing algorithms.
In a preferred embodiment, the mobile device application also has the capacity for collection of qualitative factors such as menopause symptom frequency and intensity and anthropometrics. Algorithms housed in-app or in cloud-hosted databases assess both quantitative (urine analyte) and qualitative (symptom) factors for personalized metrics (e.g. symptom/analyte correlations, symptom/intervention/analyte correlations, etc.)

In accordance with one aspect of the present invention, disclosed herein is a device for measuring analytes in a urine sample, wherein the device comprises a cassette comprising a plurality of lateral flow or dry chemistry assays, wherein a first assay of the plurality of assays is configured to measure a measured urine concentration corresponding to the concentration or dilution of the urine sample, wherein a second assay of the plurality of assays is configured to measure a first analyte reading of a first analyte, wherein the first analyte reading is normalized based upon the measured urine concentration.

In some aspects, the first analyte is an indicator of or relates to symptoms of one or more of the following: menopausal transition; osteopenia, bone loss, reduced bone mass density or osteoporosis; Polycystic Ovarian Syndrome; cancer (including ovarian cancer, breast cancer, pancreatic cancer, leukemia, non-small-cell lung cancer, colorectal cancer); fertility, pregnancy or postpartum depression; depression or major depressive disorder; menstruation, menstrual cramps, primary dysmenorrhea or endometriosis; fetal abnormalities; inflammatory and autoimmune diseases (chronic inflammation, Type II diabetes, arteriosclerosis, asthma or thyroid disease); thyroid function; cardiovascular function (including cardiovascular disease, oxidative stress, angina, adverse cardiovascular events or ischemic heart failure); kidney function (chronic kidney disease); gastrointestinal function (Crohn's disease, ulcerative colitis or inflammatory bowel disease); liver function; bladder & urine problems (bladder pain, malodorous urine, urinary tract infections, overactive bladder syndrome, bladder pain syndrome, uncomplicated cystitis, uncomplicated pyelonephritis, urosepsis or asymptomatic bacteriuria); sexually transmitted infections; migraine or headache; and toxin exposure or toxicology testing.

In some aspects, the first analyte is a hormone or metabolite that is an indicator of or relates to symptoms of menopausal transition.

In some aspects, the first analyte may be selected from the group: estrone-3-glucoronide (E1g), estrogen, estriol or estrone metabolites; Pregnanediol (PdG) or progesterone metabolites; Follicle Stimulating Hormone (FSH); Epinephrine; norepinephrine; cortisol; 17-hydroxyprogesterone; 2-hydroxyestrone; Kynurenine; tryptophan; fumarate; malate; trimethylamine; putrescine; succinate; Taurine; etiocholan-3α-ol-17-one sulfate; 2-oxoglutarate; phenylacetylglutamine; testosterone or testosterone metabolites; Dehydroepiandrosterone sulfate (DHEA-S); Sex-hormone binding globulin; inhibin A; inhibin B; melatonin; 6-sulfatoxymelatonin; putrescine; and nitric oxide metabolites.

In accordance with another aspect of the present invention, the device additionally comprises a third assay configured to measure a second analyte reading of a second analyte, wherein the second analyte reading is normalized based upon the measured urine concentration.

In some aspects, the first analyte and the second analyte are indicators of or relate to symptoms of one or more of the following: menopausal transition; osteopenia, bone loss, reduced bone mass density or osteoporosis; Polycystic Ovarian Syndrome; cancer (including ovarian cancer, breast cancer, pancreatic cancer, leukemia, non-small-cell lung cancer, colorectal cancer); fertility, pregnancy or postpartum depression; depression or major depressive disorder; menstruation, menstrual cramps, primary dysmenorrhea or endometriosis; fetal abnormalities; inflammatory and autoimmune diseases (chronic inflammation, Type II diabetes, arteriosclerosis, asthma or thyroid disease); thyroid function; cardiovascular function (including cardiovascular disease, oxidative stress, angina, adverse cardiovascular events or ischemic heart failure); kidney function (chronic kidney disease); gastrointestinal function (Crohn's disease, ulcerative colitis or inflammatory bowel disease); liver function; bladder & urine problems (bladder pain, malodorous urine, urinary tract infections, overactive bladder syndrome, bladder pain syndrome, uncomplicated cystitis, uncomplicated pyelonephritis, urosepsis or asymptomatic bacteriuria); sexually transmitted infections; migraine or headache; and toxin exposure or toxicology testing.

In some aspects, the first analyte and the second analyte is a hormone or metabolite that is an indicator of or that relates to symptoms of menopausal transition.

In some aspects, the first analyte and the second analyte may be selected from the group: estrone-3-glucoronide (E1g), estrogen, estriol or estrone metabolites; Pregnanediol (PdG) or progesterone metabolites; Follicle Stimulating Hormone (FSH); epinephrine; norepinephrine; cortisol; 17-hydroxyprogesterone; 2-hydroxyestrone; kynurenine; tryptophan; fumarate; malate; trimethylamine; putrescine; succinate; taurine; Etiocholan-3α-ol-17-one sulfate; 2-oxoglutarate; Phenylacetylglutamine; testosterone or testosterone metabolites; Dehydroepiandrosterone sulfate (DHEA-S); Sex-hormone binding globulin; inhibin A; inhibin B; melatonin; 6-sulfatoxymelatonin; putrescine; and nitric oxide metabolites.

In yet another aspect, disclosed herein is a system for measuring and monitoring analytes in a urine sample, comprising the aforementioned device and a reader unit configured to quantitatively measure the first analyte reading (and second analyte reading, where applicable) and to normalize each analyte reading based upon the measured urine concentration.

In yet another aspect, also disclosed herein is a device for measuring analytes in a urine sample, wherein the device comprises a cassette comprising a plurality of lateral flow or dry chemistry assays, wherein a first assay of the plurality of assays is configured to measure a first analyte reading of a first analyte, wherein a second assay of the plurality of assays is configured to measure a second analyte reading of a second analyte, and wherein the second analyte reading is normalized based upon the first analyte reading.

Also disclosed herein is a corresponding method for measuring and analytes (including those relating to symptoms of menopause transition) in a urine sample.

### BRIEF DESCRIPTION OF THE DRAWINGS

Embodiments of the present invention are described below with reference to the accompanying drawings in which:

FIG. 1 is an illustration of an exemplary cassette in accordance with an aspect of the present invention.

FIG. 2 is a diagrammatic representation illustrating the configuration of a lateral flow strip for carrying out a lateral flow assay, in accordance with an aspect of the present invention.

FIG. 3 is a diagrammatic representation showing the configuration of the normalization assay (as a side and corresponding top view), in accordance with an aspect of the present invention.

FIGS. 4A, 4B and 4C are a set of plots representing inter- and intra-individual variation urinary water content and illustrating the impact of normalizing for these variations.

FIG. 5 is a flowchart illustrating data acquisition and processing by the system of the present invention.

### DETAILED DESCRIPTION OF THE INVENTION

A preferred embodiment of the present invention is described and illustrated herein. A cassette houses and is configured with two or more colorimetric or fluorometric assays (lateral flow and/or dry chemistry assays). One or more of the assays is configured to measure the concentration of specific target analytes (such as a specific hormone or metabolite) from urine collected on an exposed sample pad. In the preferred embodiment, the target analyte (or analytes) relates to symptoms of menopausal transition. One of the other assays (the normalization assay) is configured to quantify the urine dilution or concentration relative to population standards; this enables the normalisation of the quantitative results of the one or more target analytes to account for differences in urine concentration. The cassette is configured to work in conjunction with a reader unit (which may be a hand-held or a countertop device). The reader unit is configured to measure/analyse the concentration of the analytes. The cassette and reader unit are preferably designed and intended for at-home use, although they may of course also be used outside the home, such as at medical facilities, clinics, etc.

LFA Platform: Cassette. The cassette is configured to house one or more multiplex lateral flow assays for fluorescent detection, such as:
Assay 1: Estrone-3-glucuronide (E3g) and Luteinizing Hormone (LH).
Assay 2: Pregnanediol (PdG) and Follicle Stimulating Hormone (FSH).
Normalization array: a colorimetric assay for normalization of urine concentration such as Creatinine (Cr) or specific gravity.

The quantitation of two or more analytes is desirable for improved event identification and prediction due to the dynamic relationships between circulating hormones. For example, hormones relevant to the menstrual cycle and menopause transition are a part of three feedback loops, via the hypothalamus, anterior pituitary, ovaries and endometrium. Some symptoms during menopause transition are hypothesized to be related to levels of hormones in combination, rather than the level or rate of change of a single hormone; for example, low urinary estrogen levels with higher urinary FSH and norepinephrine levels are associated significantly with high severity hot flashes along with moderate sleep, mood, cognitive and pain symptoms. Multivariate quantitation also improves predictive power in alternate embodiments. For example, prostate cancer screening via urine quantification of two or more urinary biomarkers, e.g. pigment epithelium-derived factor, hemopexin, calnexin precursor, hornerin, keratin 13, etc., has a demonstrably higher performance than traditional measurement of prostate-specific antigen in serum. Multivariate assessment of urinary hippurate, formate and 4-cresol sulfate in can distinguish between inflammatory bowel disease, Crohn's disease and ulcerative colitis, with implications for diagnosis and disease management.

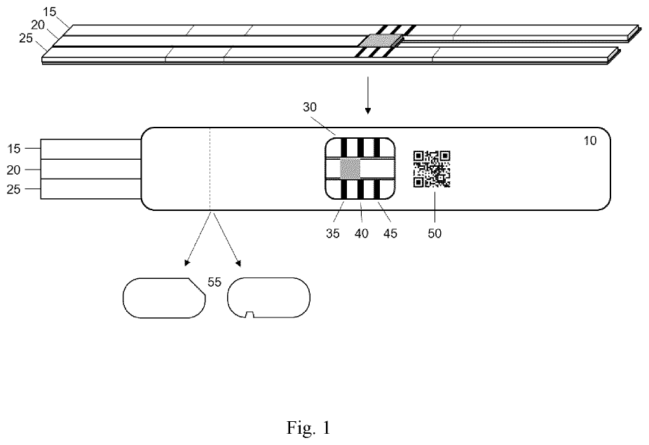

Referring to FIG. 1, this illustrates a cassette 10, housing several multiplex lateral flow assays, namely Assay 1 (15), Assay 2 (25), and a normalization assay 20.

The cassette includes:
A test window 30 over the region of interest (test lines 35, 40 and control lines 45 for the lateral flow assays).
Staggered vertical support features to maintain proper positioning of assay strip components within the cassette.
An identifying mark or feature such as a barcode or QR code 50 for assay lot and calibration curve information.
Physical design characteristics such an alignment groove 55 or an unsymmetrical cross-section to ensure correct orientation for reader orientation.

It is contemplated that in some aspects, the components which make up the cassette may be single-use and disposable. In yet other aspects, some of the components may be reusable.

When the cassette/device is to be used, urine is collected on the sample pad of the cassette by exposing the sample pad of the cassette to midstream urine or by dipping it into a collected container of urine. After collecting urine on the sample pad, the opposite end of the cassette portion of the device is inserted into the reader unit, triggering a countdown. The urine sample moves through the assay by capillary action, or wicking. The sample pad is treated to neutralize the pH of the urine and to remove physical contaminants (crystals, cells). At the conjugate pad, analytes of interest in the sample interact with antibody-label conjugates either competitively or indirectly (sandwich assay); the results are read at test lines specific for the analyte on a nitrocellulose membrane; a control line on each assay confirms that the assay performed properly and appropriately; a wick pad draws off the remainder of the sample. The analytes and detection ranges are calibrated for known correlations with the menopausal transition and perimenopausal symptoms with additional range for expected differences in urine concentration.

For the normalization assay, the urine sample moves along a sample pad to a treated membrane. The normalization analyte and detection range is designed to accommodate the expected variability of urine concentration fluctuations in the target population.

A colorimetric or fluorometric measurement of each assay occurs automatically at a pre-specified time point following insertion into the reader. The appropriate calibration curve for each assay is determined based on analyte and manufacturing lot based on the QR code, and this equation is used to convert the colorimetric or fluorometric readings to a concentration. Finally, the hormone and metabolite concentrations are normalized (divided by) the creatinine concentration to produce a final set of normalized values. This information is relayed to the user. The reader unit is generally provided with communication means (wired or wireless, e.g. wi-fi, Bluetooth™, via Internet or telecommunications networks, etc.) for communicating the user's information either directly to the user (e.g. through a smartphone or computer application) or, preferably, first to a cloud database and then delivered to the user via user's smartphone application (in the latter case, the information may undergo further processing before delivery to the use in order to provide information that is more useful, user-friendly, detailed, enriched, etc.

Type of Analytes. In one preferred embodiment, the device is configured to measure/quantify an analyte such as estrone-3-glucoronide (E1g) for a menopause-relevant dynamic range (1.5-300 ng/ml). Other suitable known analytes are also possible, which may be used in place of or in combination with the above. One such alternative, for example, as mentioned is “Pregnanediol (PdG) and Follicle Stimulating Hormone (FSH)” with respective ranges of 1-32 μg/mL and 1-170 IU/L. Other alternatives include epinephrine (1-50 ng/ml), norepinephrine (4-245 ng/ml), cortisol (7-500 ng/ml), as well as related metabolites of the aforementioned analytes such as 17-hydroxyprogesterone or 2-hydroxyestrone.

### LFA Assays: Hormones and Hormone Metabolites.

Referring to FIG. 2, this is a diagrammatic representation illustrating the configuration of a lateral flow strip for carrying out a lateral flow assay. Each multiplex lateral flow strip consists of a sample pad 60, conjugate pad 65, nitrocellulose membrane 70 and wick pad 75 adhered to the backing card 80, and is run as a dipstick. The nitrocellulose membrane 70 has two test lines (TL) 35, 40 and one control line (CL) 45 for each multiplexed strip (shown as areas of overlap). The assays may use a fluorescent indicator such as europium latex particles.

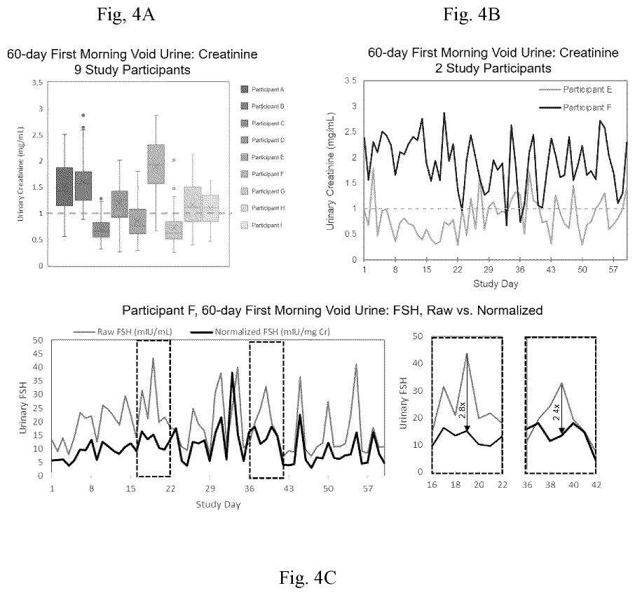

Normalization. FIGS. 4A, 4B and 4C demonstrate that there can be significant inter- and intra-individual variability in the composition of first morning void urine based on creatinine concentration from participants in a 60-day longitudinal study. The 50th percentile for creatinine concentration in US women is approximately 1 mg/mL; the 20th and 90th percentile are approximately 0.25 mg/ml and 2 mg/mL. Normalizing analyte concentrations by these values therefore scales the raw concentrations values from anywhere from one-quarter to double the original value.

FIG. 4A depicts the cumulative analysis of first morning void creatinine concentration for each of 9 study participants over a 60-day longitudinal study, illustrating the minimum, maximum, upper and lower quartiles and median for each. The range across all study participants is similar to the expected population-level values. FIG. 4B compares the day-to-day variation in first morning void creatinine concentration for two study participants, one whose average creatinine concentration was below the population 50^{th} percentile and one well above this mark. The plot shows significant variation for both, in some cases creatinine concentrations more than doubling day-over-day indicating a two-fold reduction in urinary water content. FIG. 4C compares the day-to-day raw follicle-stimulating hormone concentration for one study participant against the same values normalized by creatinine Concentration. Cursory analysis of only the raw data would indicate 7 major peaks of roughly equal magnitude in FSH concentration over the course of 60 days; in this case, normalization significantly flattens the majority of those peaks, in some cases with a 2.4 to 2.8-fold decrease. The normalized trend is left with only 1 significant peak over the study period. Analyzing the raw data may erroneously correlate false FSH spikes with symptom or trigger data.

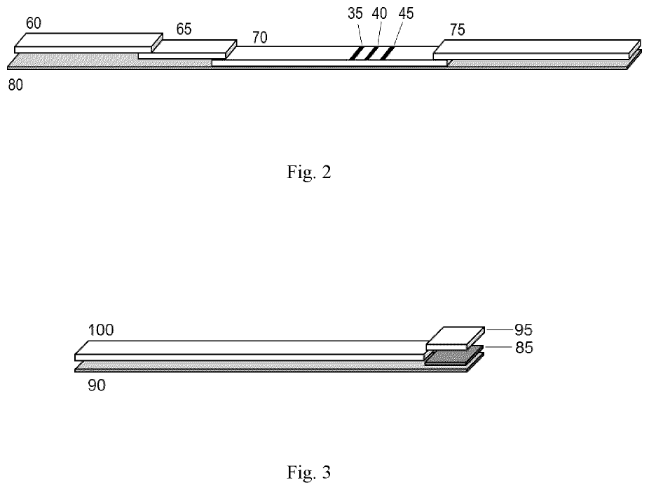

Normalization Assay: Creatinine. As an example, the normalization assay can be carried out via colorimetric quantification of a metabolite such as creatinine, which is typically excreted at a constant rate for a single individual. In its preferred embodiment, this is a dry chemistry membrane applied via an adhesive layer 85 to a plastic support 90 to create a dipstick test strip. The membrane 95 is composed of a material such as cellulose that has been coated with 2 separate solutions: a hydroxide to create an alkaline environment and a chromogen. A sample pad 100 wicks the collected sample to the treated membranes and it some embodiments pre-treats the sample with chemicals components such as a buffer to neutralize the sample pH. FIG. 3 is a diagrammatic representation showing the configuration of the normalization assay, in accordance with an aspect of the present invention. An alternate normalization mode involves the quantitation of urine specific gravity via colorimetric dry chemistry assay.

Reader. The countertop reader unit performs analysis of a urine sample via reflectance and fluorescence measurements of regions of interest (ROI) on the fluorescent and colorimetric assays. In a preferred embodiment, the software application links to an individual reader unit via an identifying code, such as a scanned QR sticker, associated with the unit. A single-use cassette is inserted into a port on the device for sample measurement, activating the reader and beginning an automatic countdown. At the programmed timepoints for the individual assays, the reader performs a colorimetric or fluorometric reading of the appropriate ROI. Calibration curve information for the assay lot is communicated to the reader via a lot number by any number of means, such as scanning a bar code or QR code on the cassette, manually inputting the lot number into the software application, reading an RFID tag embedded in the cassette, etc. With the calibration information, the processor on the reader unit converts the reflectance and fluorescence readings to concentration and uploads the data to the secure cloud-based server via Wi-Fi. A limited number of readings can be stored locally on the reader in the event that a reading occurs without Wi-Fi access. Stored concentration data can be deleted locally once it is uploaded to a secure cloud-based server. The reader includes a battery for temporary mobile functionality and in some embodiments may include a screen to communicate information such as instructions and results to the user. In the preferred embodiment, the reader includes LED lights to indicate status to the user, but results are communicated to the user only via the software application.

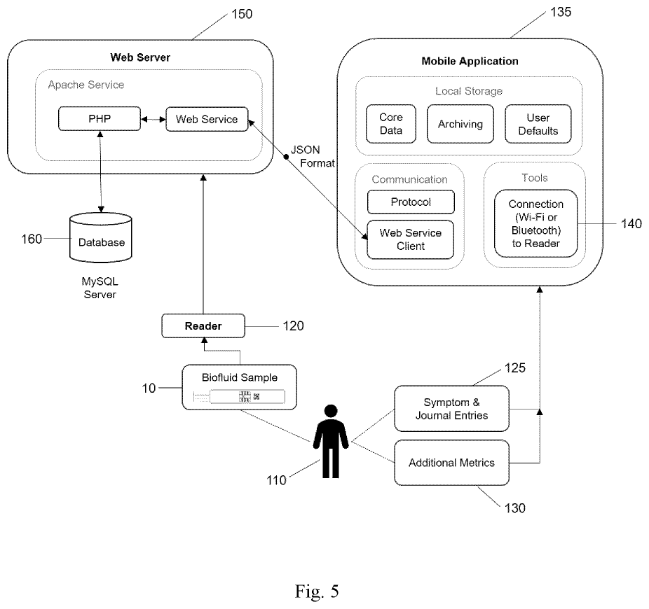

FIG. 5 is a flowchart illustrating the data acquisition and processing steps, as may be carried out by the system of the present invention. A user 110 uses a cassette/device 10 to collect a sample of urine and to perform lateral flow assays to measure certain analytes in the urine. The analytes are measured by a reader unit 120, which communicates the measured data to a mobile application 135 on the user's mobile device (such as a smartphone, tablet, laptop, etc.) via Wi-Fi or Bluetooth™. Optionally, the user may enter/upload additional information (such as symptom and journal entries 125, or additional metrics 130) to the mobile application through the user's mobile device. The measured data and additional information is communicated to a web server 150, and stored on a database 160. The measured data may be processed (e.g. calibrated by applying the above-discussed urine concentration normalization method, or calculated as ratios of two or more analytes pre- or post-normalization or in the absence of urine concentration normalization), and communicated/shared/stored and then, where applicable, presented to the user.

In one alternative embodiment, it is contemplated that the above device may be adapted to carry out the normalization step based on a second analyte, instead of based on the urine concentration. Thus, also disclosed herein is a device for measuring analytes in a urine sample, wherein the device comprises a cassette comprising a plurality of lateral flow or dry chemistry assays, wherein a first assay of the plurality of assays is configured to measure a first analyte reading of a first analyte, wherein a second assay of the plurality of assays is configured to measure a second analyte reading of a second analyte, and wherein the second analyte reading is normalized based upon the first analyte reading. By way of example, it is contemplated that in some instances, the measurement of estrogen or an analyte related to estrogen (as the second analyte) may be normalized based on the measurement of pregnanediol (as the first analyte), rather than on urine concentration.

As previously mentioned, it is understood that the disclosed device, method and system may also be applied/adapted for conditions other than menopausal transition by measuring other suitable analytes that are indicators of or associated with such conditions. Some of these examples are listed in Table 1 below.

TABLE 1

| Analyte is an indicator | |
|---|---|
| of or relates to the | |
| following conditions | Possible/Suitable Analyte |
| Osteopenia, bone | Urinary-N-telopeptide; succinate; taurine; Follicle |
| loss, reduced bone | Stimulating Hormone; melatonin; 6-sulfatoxymelatonin. |
| mass density, | |
| osteoporosis | |
| Polycystic Ovarian | Threonine; succinic acid; 2-oxoglutarate; hippuric acid; |
| Syndrome | Sex-hormone binding globulin; testosterone and |
| | testosterone metabolites; Sex-hormone binding globulin; |
| | 2-oxoglutarate (alpha-Ketoglutaric acid). |
| Cancer (including but | Acetate; urea; succinate; formate; hippuric acid; alpha- |
| not limited to ovarian, | fetoprotein; REG1A protein; TFF1 protein; LYVE1 |
| breast, pancreatic; | protein; N-ethylglycine; N1-acSpd; n8-acSd; DiAcSpm |
| leukemia, non-small- | (N1,N12-diacetylspermine); DiAcSPpd (N1,N8- |
| cell lung cancer, | diacetylspermidine); 8-OHdG. |
| colorectal cancer. | |
| Fertility, pregnancy, | Formate; succinate; glutamine; human chorionic |
| postpartum depression | gonadotropin; prolactin or lactotropin; estrone-3- |
| | glucoronide (E1g) and estrogen, estriol and estrone |
| | metabolites; Pregnanediol (PdG) and progesterone |
| | metabolites; inhibin A; Sex-hormone binding globulin; |
| | 6-sulfatoxymelatonin; formate. |
| Depression; major | Succinic acid; L-tyrosine; N-Methylnicotinamide; |
| depressive disorder; | hypoxanthine; indoxyl sulfate; L-phenylalanine; |
| | melatonin; cortisol; 6-sulfatoxymelatonin. |
| Menstruation; | Histidine; ornithine; citrulline; estrone-3-glucoronide |
| menstrual cramps; | (E1g) and estrogen, estriol and estrone metabolites; |
| primary | Pregnanediol (PdG) and progesterone metabolites; |
| dysmenorrhea; | Follicle Stimulating Hormone (FSH); Luteinizing |
| endometriosis | Hormone (LH); testosterone and testosterone |
| | metabolites; 17-hydroxyprogesterone; 2-hydroxyestrone; |
| | Dehydroepiandrosterone sulfate; prolactin; Sex-hormone |
| | binding globulin; melatonin; 6-sulfatoxymelatonin; nitric |
| | oxide metabolites; blood protein CA 125. |
| Fetal abnormalities | Inhibin A; Inhibin B; alpha-fetoprotein; invasive |
| | trophoblast antigen; β-core fragment; free β human |
| | chorionic gonadotropin (βhCG); total hCG; oestriol; |
| | gonadotropin peptide. |
| Inflammatory and | c-Reactive protein; glucose; thyroid-stimulating |
| autoimmune diseases | hormone; thyroxine; antithyroid peroxidases; |
| (Chronic inflammation; | antithyroglobulin antibodies; rheumatoid factor; anti- |
| Type II diabetes; | cyclic citrullinated peptide. |
| arteriosclerosis; | |
| asthma, thyroid | |
| disease) | |
| Thyroid function | thyroid-stimulating hormone; thyroxine; antithyroid |
| | peroxidases; antithyroglobulin antibodies; |
| | triiodothyronine; 3,5-T2 (3,5-diiodothyronine). |
| Cardiovascular | p-Cresol sulfate; trimethylamine N-oxide; 8-hydroxy-2- |
| function (including but | deoxyguanine; 11-dehydro-thromboxane B2; |
| not limited to | methylmalonic acid; cytosine; phenylacetylglycine; |
| cardiovascular | 8-OHdG |
| disease; oxidative | |
| stress; angina; | |
| adverse | |
| cardiovascular events; | |
| ischemic heart failure) | |
| Kidney function | Creatine; creatinine; trimethylamine N-oxide; DIT (3,5- |
| (chronic kidney | diiodotyrosine); thyronine; albumin. |
| disease) | |
| Gastrointestinal | Hippurate; formate; 4-cresol sulfate. |
| function (Crohn's | |
| disease; ulcerative | |
| colitis; inflammatory | |
| bowel disease) | |
| Liver function | GLCA-Sul (glycine-amidated sulfated lithocholic acid); |
| | albumin; bilirubin. |
| Bladder & urine | Malate; putrescine; Etiocholan-3α-ol-17-one sulfate; |
| problems (bladder | 2-oxoglutarate (alpha-Ketoglutaric acid); |
| pain, malodorous | Phenylacetylglutamine; pyuria; nitrite; c-Reactive |
| urine, urinary tract | protein; cytokines; neutrophil gelatinase-associated |
| infections, overactive | lipocalin (NGAL); lactoferrin; bone morphonegenic |
| bladder syndrome; | protein-2; cystatin-C; heparin binding protein; human |
| bladder pain syndrome; | defensin-5; human neutrophil peptides -1, -3; |
| uncomplicated cystitis; | lipopolysaccharide binding protein; matrix |
| uncomplicated | metalloprotease-9; procalcitonin. |
| pyelonephritis; | |
| urosepsis; asymptomatic | |
| bacteriuria) | |
| Sexually transmitted | Sucrose; Lactate; Pyruvate. |
| infections | |
| Migraine & headache | Estrogen, estriol and estradiol and their metabolites; |
| | progesterone and its metabolites; nitric oxide |
| | metabolites; thiobarbituric acid reactive substances |
| | (TBARS); albumin; 6-sulphatoxymelatonin. |
| Toxin exposure & | Muconic acid; trans, trans-muconic acid; toxic metals |
| toxicology testing | (arsenic; cadmium; cobalt; lead; mercury; thallium); 8- |
| | OHdG and DNA repair metabolites; Amphetamines (D- |
| | amphetamine; D-methamphetamine; 3,4- |
| | methylenedioxyamphetamine; 3,4- |
| | methylenedioxyamphetamine); Benzodiazepines |
| | (Diazepem metabolites nordiazepam and/or oxazepam); |
| | Cocaine and metabolites (benzoylecgonine); Opiates |
| | (diacetylmorphine; morphine; codeine;); Synthetic |
| | opiods (methadone; oxycodone; fentanyl; tramadol); |
| | Marijuana and metabolites (11-nor-9-carboxy-delta-9- |
| | tetrahydrocannabinol); Lysergic acid diethylamide (LSD); |
| | Tricyclic antidepressants (TCAs); Non-benzodiazepine |
| | hypnotics (zolpidem, zopiclone); Ketamine; Mescaline |
| | (peyote); Psilocybin; Gamma-hydroxybutyrate (GHB); |
| | 1,4-Butanediol (precursor to GHB); Chloral hydrate; |
| | Synthetic/designer cannabinoids (‘spice’ and ‘K2’); |
| | Tryptamines; Phenethylamine derivatives (synthetic |
| | stimulants, ‘bath salts', ‘2C’ drugs); Imidazoline receptor |
| | agonists (clonidine, tetrahydrozoline, oxymetazoline); |
| | chlorinated phenols (mono-, di- tri- and tetra- and |
| | pentachlorophenol); phenol; p-Cresol; catechol; |
| | hydroquinones; benzenediols; p-Nitrophenol; herbicides |
| | and pesticides (2,4-D, 2,4,5-T, sivex, dicamba); |
| | phthalate esters; hydroxyl polycyclic aromatic |
| | hydrocarbons (OH-PAHs); brominated phenols (BRPs); |
| | 5 hydroxyl polybrominated diphenyl ethers (OH-PBDEs); |
| | triclosan; tetrabromobisphenol A. |

While specific embodiments have been described and illustrated, such embodiments should be considered illustrative of the invention only and not as limiting the invention as construed in accordance with the accompanying claims. The described features, structures, or characteristics may be combined in any suitable manner in one or more embodiments. One skilled in the relevant art will recognize, however, that the invention can be practiced without one or more of the specific details, or with other methods, components, materials, etc. In other instances, well-known structures, materials, or operations are not shown or described in detail to avoid obscuring aspects of the invention.

1. A device for measuring analytes in a urine sample, wherein the device comprises a cassette comprising a plurality of lateral flow or dry chemistry assays,
wherein a first assay of the plurality of assays is configured to measure a measured urine concentration corresponding to the concentration or dilution of the urine sample; and
wherein a second assay of the plurality of assays is configured to measure a first analyte reading of a first analyte, wherein the first analyte reading is normalized based upon the measured urine concentration.

2. The device of claim 1, wherein the first analyte is an indicator of or relates to symptoms of selected from the group consisting of: (menopausal transition; osteopenia, bone loss, reduced bone mass density or osteoporosis; Polycystic Ovarian Syndrome; cancer (including ovarian cancer, breast cancer, pancreatic cancer, leukemia, non-small-cell lung cancer, colorectal cancer); fertility, pregnancy or postpartum depression; depression or major depressive disorder; menstruation, menstrual cramps, primary dysmenorrhea or endometriosis; fetal abnormalities; inflammatory and autoimmune diseases (chronic inflammation, Type II diabetes, arteriosclerosis, asthma or thyroid disease); thyroid function; cardiovascular function (including cardiovascular disease, oxidative stress, angina, adverse cardiovascular events or ischemic heart failure); kidney function (chronic kidney disease); gastrointestinal function (Crohn's disease, ulcerative colitis or inflammatory bowel disease); liver function; bladder & urine problems (bladder pain, malodorous urine, urinary tract infections, overactive bladder syndrome, bladder pain syndrome, uncomplicated cystitis, uncomplicated pyelonephritis, urosepsis or asymptomatic bacteriuria); sexually transmitted infections; migraine or headache; and toxin exposure or toxicology testing).

3. The device of claim 2, wherein the first analyte is an indicator of or relates to symptoms of menopausal transition.

4. The device of claim 3, wherein the first analyte is selected from the group consisting of: (estrone-3-glucoronide (E1g), estrogen, estriol or estrone metabolites; Pregnanediol (PdG) or progesterone metabolites; Follicle Stimulating Hormone (FSH); epinephrine; norepinephrine; cortisol; 17-hydroxyprogesterone; 2-hydroxyestrone; kynurenine; tryptophan; fumarate; malate; trimethylamine; putrescine; succinate; taurine; Etiocholan-3a-ol-17-one sulfate; 2-oxoglutarate; Phenylacetylglutamine; testosterone or testosterone metabolites; Dehydroepiandrosterone sulfate (DHEA-S); Sex-hormone binding globulin; inhibin A; inhibin B; melatonin; 6-sulfatoxymelatonin; putrescine; and nitric oxide metabolites).

5. The device of claim 1, wherein a third assay of the plurality of assays is configured to measure a second analyte reading of a second analyte, and wherein the second analyte reading is normalized based upon the measured urine concentration.

6. The device of claim 5, wherein the first and second analytes are indicators of or relate to symptoms of selected from the group consisting of: (menopausal transition; osteopenia, bone loss, reduced bone mass density or osteoporosis; Polycystic Ovarian Syndrome; cancer (including ovarian cancer, breast cancer, pancreatic cancer, leukemia, non-small-cell lung cancer, colorectal cancer); fertility, pregnancy or postpartum depression; depression or major depressive disorder; menstruation, menstrual cramps, primary dysmenorrhea or endometriosis; fetal abnormalities; inflammatory and autoimmune diseases (chronic inflammation, Type II diabetes, arteriosclerosis, asthma or thyroid disease); thyroid function; cardiovascular function (including cardiovascular disease, oxidative stress, angina, adverse cardiovascular events or ischemic heart failure); kidney function (chronic kidney disease); gastrointestinal function (Crohn's disease, ulcerative colitis or inflammatory bowel disease); liver function; bladder & urine problems (bladder pain, malodorous urine, urinary tract infections, overactive bladder syndrome, bladder pain syndrome, uncomplicated cystitis, uncomplicated pyelonephritis, urosepsis or asymptomatic bacteriuria); sexually transmitted infections; migraine or headache; and toxin exposure or toxicology testing).

7. The device of claim 6, wherein the first and second analytes are indicators of or relate to symptoms of menopausal transition.

8. The device of claim 7, wherein the first analyte and second analytes are selected from the group consisting of: (estrone-3-glucoronide (E1g), estrogen, estriol or estrone metabolites; Pregnanediol (PdG) or progesterone metabolites; Follicle Stimulating Hormone (FSH); epinephrine; norepinephrine; cortisol; 17-hydroxyprogesterone; 2-hydroxyestrone; kynurenine; tryptophan; fumarate; malate; trimethylamine; putrescine; succinate; taurine; Etiocholan-3a-ol-17-one sulfate; 2-oxoglutarate; Phenylacetylglutamine; testosterone or testosterone metabolites; Dehydroepiandrosterone sulfate (DHEA-S); Sex-hormone binding globulin; inhibin A; inhibin B; melatonin; 6-sulfatoxymelatonin; putrescine; and nitric oxide metabolites).

9. A system for measuring analytes in a urine sample, comprising:
a device in accordance with claim 1; and
a reader unit configured to quantitatively measure the first analyte reading and normalize the first analyte reading based upon the measured urine concentration.

10. A system for measuring analytes in a urine sample, comprising:
a device in accordance with claim 5; and
a reader unit configured to quantitatively measure the first analyte reading and the second analyte reading, and to normalize the first analyte reading and second analyte reading based upon the measured urine concentration.

11. A system for measuring analytes in a urine sample comprising:
a device in accordance with claim 5; and
a reader unit configured to quantitatively measure the first analyte reading and the second analyte reading, and configured to determine a first parameter based upon the first analyte reading and the second analyte reading both without normalization based upon measured urine concentration, and to determine a second parameter based upon the first analyte reading and the second analyte reading both with normalization based upon the measured urine concentration.

12. A device for measuring analytes in a urine sample, wherein the device comprises a cassette comprising a plurality of lateral flow or dry chemistry assays,
wherein a first assay of the plurality of assays is configured to measure a first analyte reading of a first analyte; and
wherein a second assay of the plurality of assays is configured to measure a second analyte reading of a second analyte, wherein the second analyte reading is normalized based upon the first analyte reading.
